SEQUENCE LISTING (36 sequences)

SEQ ID NO: 1 - DNA, 24 bp, Artificial
  specific primer of CITED2
tcttaactttttattgacattggc

SEQ ID NO: 2 - DNA, 22 bp, Artificial
  specific primer of CITED2
ttcaacatttttgacactgtgc

SEQ ID NO: 3 - DNA, 20 bp, Artificial
  specific primer of MGST1
aatgggtttaccccagttca

SEQ ID NO: 4 - DNA, 20 bp, Artificial
  specific primer of MGST1
catggcttacaggttgctga

SEQ ID NO: 5 - DNA, 20 bp, Artificial
  specific primer of TCL1A
tatacctgtgcctcggcttt

SEQ ID NO: 6 - DNA, 20 bp, Artificial
  specific primer of TCL1A
tgtttacatgcagccgtagc

SEQ ID NO: 7 - DNA, 18 bp, Artificial
  specific primer of CD19
ctggcccatcgggattat

SEQ ID NO: 8 - DNA, 20 bp, Artificial
  specific primer of CD19
gacaatggatttcctgagcc

SEQ ID NO: 9 - DNA, 20 bp, Artificial
  specific primer of TCF7
agcttgcaagaagcaggaag

SEQ ID NO: 10 - DNA, 20 bp, Artificial
  specific primer of TCF7
taggctggtgggtttctcag

SEQ ID NO: 11 - DNA, 20 bp, Artificial
  specific primer of TRB
aaccacttccgctgtcaagt

SEQ ID NO: 12 - DNA, 20 bp, Artificial
  specific primer of TRB
gctggtaggacaccgaggta

SEQ ID NO: 13 - DNA, 22 bp, Artificial
  specific primer of RRAGB
gatgaattgttcatgaagtcgg

SEQ ID NO: 14 - DNA, 20 bp, Artificial
  specific primer of RRAGB
caagatgatgccgagaaagg

SEQ ID NO: 15 - DNA, 22 bp, Artificial
  specific primer of DCK
tccaagtcatttttgtttttgg

SEQ ID NO: 16 - DNA, 25 bp, Artificial
  specific primer of DCK
aaaaatgcatacgtataatgagaca

SEQ ID NO: 17 - DNA, 899 bp, Homo sapiens
gagggcaacggaggggaaataaaagggaacggctccgaatcggccccagcggccgctgcgagacctcggcgccgacatcgcgacagaagcgctttgcacgccaggaaggtcccctctatgtgctgctgagccggtcctggacgcgacgagcccgccctcggtcttcggagcagaaatcgcaaaaacggaaggactggaaatggcagaccatatgatggccatgaaccacgggcgcttccccgacggcaccaatgggctgcaccatcaccctgcccaccgcatgggcatggggcagttcccgagcccccatcaccaccagcagcagcagccccagcacgccttcaacgccctaatgggcgagcacatacactacggcgcgggcaacatgaatgccacgagcggcatcaggcatgcgatggggccggggactgtgaacggagggcaccccccgagcgcgctggcccccgcggccaggtttaacaactcccagttcatgggtcccccggtggccagccagggaggctccctgccggccagcatgcagctgcagaagctcaacaaccagtatttcaaccatcacccctacccccacaaccactacatgccggatttgcaccctgctgcaggccaccagatgaacgggacaaaccagcacttccgagattgcaaccccaatgtcatagacactgatttcatcgacgaggaagttcttatgtccttggtgatagaaatgggtttggaccgcatcaaggagctgcccgaactctggctggggcaaaacgagtttgattttatgacggacttcgtgtgcaaacagcagcccagcagagtgagctgttgactcgatcgaaaccccggcgaaagaaatcaaacccccaacttcttcggcgtgaattaaa

SEQ ID NO: 18 - DNA, 541 bp, Homo sapiens
gaattcaagtcctaaagcctacagttttgaatactactgaaatgacaagttattccagaccaaaattgaaaaaatggttgacctcacccaggtaatggatgatgaagtattcatggcttttgcatcctatgcaacaattattctttcaaaaatgatgcttatgagtactgcaactgcattctatagattgacaagaaaggtttttgccaatccagaagactgtgtagcatttggcaaaggagaaaatgccaagaagtatcttcgaacagatgacagagtagaacgtgtacggagagcccacctgaatgaccttgaaaatattattccatttcttggaattggcctcctgtattccttgagtggtcccgacccctctacagccatcctgcacttcagactatttgtcggagcacggatctaccacaccattgcatatttgacaccccttccccagccaaatagagctttgagtttttttgttggatatggagttactctttccatggcttacaggttgctgaaaagtaaattgtacctgtaa

SEQ ID NO: 19 - DNA, 2206 bp, Homo sapiens
cgcggggccagggcggcggcccccagggagttggcaggatggcagagggcaaggcaggcggcgcggccggcctcttcgccaagcaggtgcagaagaagtttagcagggcccaggagaaggtgctgcagaaattggggaaagctgtagaaaccaaagatgaacgatttgaacaaagcgctaacaacttctaccaacaacaggcagaaggccacaagctgtacaaggacctgaagaacttccttagtgcagtcaaagtgatgcatgaaagttcaaaaagagtgtcagaaaccctgcaggagatctacagcagcgagtgggatggtcatgaggagctgaaggccatcgtatggaataatgatctcctttgggaagactacgaggagaaactggctgaccaggctgtaaggaccatggaaatctatgttgcccagttcagtgaaattaaggagagaattgccaagcggggtcggaaactcgtggactatgacagtgcccgacaccacctggaggcagtgcagaatgccaagaagaaagatgaggccaagactgccaaggcagaggaagagttcaacaaagcccagactgtgtttgaagatctgaaccaagaactactagaggagctgcctattctttataatagtcgtattggctgctatgtgaccatcttccaaaacatttccaacttgagggatgtcttctacagggaaatgagcaagctgaaccacaatctctacgaggtgatgagcaaactggagaagcaacattccaataaagtctttgtggtgaagggactgtcaagcagcagcaggcgctctttagtcatttctcccccagttcgaacagctacagtctccagtcctcttacctcacctactagtccctctacactttccttgaagagtgagagtgaatctgtctcagcaactgaagatctggcacctgatgcagcccaaggggaagacaattctgagatcaaggagctcttagaagaggaggaaatagagaaggaaggatctgaagcaagctcctctgaggaagatgagcctctaccagcctgcaatggccccgcccaggcccagccctctcctaccactgaaagggccaagtcccaggaggaagttctccccagctccacaactccatcaccaggcggagccctgagcccttcagggcagccttcatcatctgccacagaagtagtcctccgaacccgcaccgcaagtgaaggatctgaacaaccaaagaagagagcctctatccagaggacctcagcaccccctagtaggcctcctccacccagagccactgcaagccccaggccctcctcagggaacataccttccagccctacagcctctggagggggttcacccaccagccctagggcctccttggggactgggactgcaagtcctaggacctccctagaggtctctcctaatccagaaccaccagagaagccagtaagaactcctgaggccaaagaaaatgaaaacatccacaatcagaaccctgaagaactttgtacttcccccaccttaatgacatctcaggttgcttcagagcctggagaggcaaagaagatggaagacaaggaaaaggataataagcttatctcagctgactcctcggagggccaagaccagcttcaagtctccatggtaccagaaaacaacaacctcacagcacctgaacctcaagaagaggtatccacaagtgaaaatccacaactctgaagagaaactaccaagactcctcctgccccaaacctcgccagagaagctcttcaaccagagggtataggtcagagggatataagagccagcatccatccctgggttctcagtaggaatgctggtgctgtctaaagacctggcattaatggaggcggaggagcagccttacgggagggatggagggaggcaggctggggagaagagaacattagactcagggaatatttaattctggttttagcattattagaataagactttatacattaactaaagtggagctttaatcactataaaaagcaaaagtatctatagacacagacacttgcctatacagagacataaccacacacactcagaggatagtgaacaaatctgtctttgacttacgacccattttgcaagacttaaagccggaagaacacattttcagattgttaaataaagtctgattctgactaaaaaaaaaa

SEQ ID NO: 20 - DNA, 912 bp, Homo sapiens
ggcacgagcgctcatggcgggcggaggagccggggaccccggcctgggggcggccgccgccccagcgcccgagacccgcgagcacctcttcaaggtgctggtgatcggcgagcttggcgtgggcaagaccagcatcatcaagcgctacgtccaccagctcttctcccagcactaccgggccaccatcggggtggacttcgccctcaaggtcctcaactgggacagcaggactctggtgcgcctgcagctgtgggacatcgcggggcaggagcgatttggcaacatgacccgagtatactacaaggaagctgttggtgcttttgtagtctttgatatatcaagaagttccacatttgaggcagtcttaaaatggaaaagtgatctggatagtaaagttcatcttccaaatggcagccctatccctgctgtcctcttggctaacaaatgtgaccagaacaaggacagtagccagagtccttcccaggtggaccaattctgcaaagaacatggctttgccggatggtttgaaacctctgcaaaggataacataaacatagaggaagctgcccggttcctagtggagaagattcttgtaaaccaccaaagctttcctaatgaagaaaacgatgtggacaaaattaagctagatcaagagaccttgagagcagagaacaaatcccagtgttgctgatatatggcttctgcttctcttgtgtgtgcctcagctctgaagaagttcctgagaatgggttacagatgtcatgttagctgggagtcttcccacatgtggcacttcaaaaggcagcaccactgggcgcctgcacttatttgaaaatggaactttgggagaagtatccctgctagtggctctgtaacttaacagatgacaattaggcttttgtcattgttgcc

SEQ ID NO: 21 - DNA, 1817 bp, Homo sapiens
ctgtcagaatggccaccatggtaccatccgtgttgtggcccagggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacgggctcccggagcgtgtggagctggcacccctgcctccttggcagccggtgggccagaacttcaccctgcgctgccaagtggagggtgggtcgccccggaccagcctcacggtggtgctgcttcgctgggaggaggagctgagccggcagcccgcagtggaggagccagcggaggtcactgccactgtgctggccagcagagacgaccacggagcccctttctcatgccgcacagaactggacatgcagccccaggggctgggactgttcgtgaacacctcagccccccgccagctccgaacctttgtcctgcccgtgacccccccgcgcctcgtggccccccggttcttggaggtggaaacgtcgtggccggtggactgcaccctagacgggctttttccagcctcagaggcccaggtctacctggcgctgggggaccagatgctgaatgcgacagtcatgaaccacggggacacgctaacggccacagccacagccacggcgcgcgcggatcaggagggtgcccgggagatcgtctgcaacgtgaccctagggggcgagagacgggaggcccgggagaacttgacggtctttagcttcctaggacccattgtgaacctcagcgagcccaccgcccatgaggggtccacagtgaccgtgagttgcatggctggggctcgagtccaggtcacgctggacggagttccggccgcggccccggggcagccagctcaacttcagctaaatgctaccgagagtgacgacggacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgagacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcaccaacggagcggcagttaccatgttagggaggagagcacctatctgcccctcacgtctatgcagccgacagaagcaatgggggaagaaccgtccagagctgagtgacgctgggatccgggatcaaagttggcgggggcttggctgtgccctcagattccgcaccaataaagccttcaaactccctaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - DNA, 1877 bp, Homo sapiens
ctgagcagagtggacgaggccttctacctcctgtagcccccagctggcttgatttggctggtcttggggtgatgcctgacaccttcaactcaaggtctccctctgtggagggttctctgtgggcagtgggcacagagagtcagggtcgagattggaggcacctgccgaccatcacaagtgagctctctggggctccccgctgccacactgtaccctgtgctgggagcacagctctccaagagcctggggagccccaggggccaccagccagcccttcgtgcccagaggaggccttggctgccacatgggagcagccatgggcttcggaggtattcgggcctgagagaatgcccccctctggagctgctcgaagcttccaagaagtaacagagccagctgtagtggcagtggaccggcaggccatcttcccagatacctgggactctcacggaggaacatggcctacagcaggagaggccaaggccagagccagggaggctgggaagcagctcccctgcctcagttaccacggagcagctaggtgcaaagatgaccgagaggggaagtgtggccaggccaacccagggacctgaaaccccaaggagcccagagggcaccacggaagctgccacccaggacgggaaggaacagccagagcttccatgtgccatggccatgggcacacccagcaccacggagaggatttccacctctggccaggcaggcaccagctcgcatggctgctgggcatggccattggcgggtctgagtgtgggccgctcctcgctaactgcatgcagccttccactctgcgcatgtctgcttgggcagaaaatgctgagacactgtggccggacctgacagtcagcacttggcagtgggctctgtggacccagcatttctcatagcgtcaacccacctcttgccttgttgaggctttttcctttcagatgagctgtccttgacattctgatgtggtgaaatggttagcagcatggactttggaaccagatagacctagatacaaaccacatgtctaacacttgctacctctgtgaccttgggcaaccccctccctcagcctcagttttctcatctgtaaagtggggagaatttccatcaagattgtccggagacctaaatgagataattcctggaaagtgtttagaacacagcatgtggtccctcaataggccgtggctgacagtacttttattacttgctttttgcttaaattaaaactttatgccgggccaggtgtggtggctcacgcctgtaatcccagcactttgggaggccgaggcgggcggatcacctgaggttgggagttcgaggccagcctgaccaacatggagaaaccccatctctactaaagatacaaaaagattagctgggcatggtggcgcatgcctgtaatcccagctactcaggaggctgaggcaggagaatcacttgaacccaggacgcagaggttgcggtgagccaagatcataccattgcactccagcctgggcaacaagagtgaaacttggtctcaaaaaacaaaaacaaaacaacaacaacaacaaaaaactttatgcctcaccctggcacagtagcttatacctgtaatcctagcactttgggaggctgaggcaagaggatcgcttgagcccaggagtttgagaccagcctaggcaacacagtgaaaccctgtctctacaaaaaattaaagattagctgggcgtggttgcacatgcctatagtcccagctgctggggaggctaaagcaggaggatggctttagcccaggagtttgaggctgcggtgagttgtgatcgcaccactgcattcagcctgagcaacagatcaagacactgtctcaaaaaaaaaaaaaa

SEQ ID NO: 23 - DNA, 453 bp, Homo sapiens
tgaagctgtacccaatttaattccataatcaatctatcaattacagtccgtccaccacctccctgtggactgtcctgagctctgttgggttcctgggatggaatcagtgcatcataaagggcattctttaagcagagaaggggccaggccaccccattcaggaactgctgcgggaataaagtgctaacttgcccccaggctgtctatgggagaccctgggcccagtctgggatgtacagggctctgggaagggggcagtcctggcggcagaacccggcctgcaggggcactttgcttagaagaggactctcctagcgggagaggctgggaggggctgcatcaggccgtggagctggttgctgtggtcatcagtatggctgcttgttcaggaagcgggagaacatggtgaaggcagcgaggggcttgtcggtgggaacatgtggccggcgccct

SEQ ID NO: 24 - DNA, 2126 bp, Homo sapiens
gttgttctcggcgggctgtggggcctccgcgccgcggccgttagtcatgtcggattctggaagttacggtcagtctgggggtgagcagcaaagttattctacctatggaaatccaggcagccaaggctatggacaagcatcacaaagctattctggctatgggcaaacgactgattcctcttatggacagaactacagcggttactccagttatggacaaagttattcacagtcctatggtggttatgagaatcaaaagcagagctcatatagccagcaaccatataataaccagggacagcagcaaaacatggaatcatcaggaagccaaggtggaagagcaccttcctatgaccagccagactatggtcaacaagattcatatgaccagcagtcaggctatgatcaacatcaaggctcatatgatgagcagtcaaattatgatcagcagcatgattcctatagtcaaaaccagcagtcctatcattcacaaagggaaaactacagccaccacacacaagatgaccgtcgtgatgtgagtaggtatggagaagataatagaggatatggcgggtcacagggaggaggtagagggcgtgggggatatgacaaggatggaagaggtcctatgacaggatcaagtggtggtgaccgcggtggcttcaaaaattttggtggtcacagggattatggacccagaacagatgctgattcagaatctgataattcagataacaacacaatctttgtgcaaggacttggggagggtgtgtctacagatcaagttggggagttctttaaacaaataggaattatcaagacaaataagaagaccggaaaaccaatgataaatctttatacagacaaggacacaggaaagccaaagggggaggcaacagtgtcatttgatgaccctccttcagctaaggcagccattgactggtttgatggaaaagaattccatggcaacatcattaaagtgtcctttgccactagaagacctgaattcatgagaggaggtggaagtggaggtgggcggcgaggccgtggaggatatagaggtcgtggaggctttcaagggagaggtggagaccccaaaagtggggattgggtttgccctaatccgtcatgcggaaatatgaactttgctcgaaggaattcctgcaatcagtgcaatgagcctagaccagaggactctcgtccctcaggaggagatttccgggggagaggctacggtggagagaggggctacagaggtcgcgggggcagaggtggagaccgaggcggctatggtggagacagaagtgggggtggctatggtggagacagaagcagcggtggtggctacagcggagatagaagtgggggcggctatggtggagacagaagtgggggtggctatggtggggacagaggcggcggctatggtggggacagaggaggcggctatggaggagaccgaggaggtggctatggaggagatcgaggtggctatggaggagaccgaggtggaggctatggtggagaccgaggaggctatggaggagatcgaggaggttacggaggagatcgaggaggttatggaggagatcgaggaggctatggaggagacagaagccgggggggctatggaggagaccgtggtggtggcagtggctacggtggagaccgaagtggaggctatggaggagacaggagtggtggcggctatggaggagaccgaggtgggggctacggaggagaccgaggtggctatggaggcaaaatgggaggaagaaacgactacagaaatgatcagcgcaaccgaccatactgatgactgttttgaatgttcctttgtctctgacatgatccatagtgaaattgccagagttttgcctggtgctctcctcgtggcctcttcttgggtagtgaaattaagtgacatttggatttttatttgggtgggagggctgggacagtttttcttctagaaatgtctgttgagatttccccctttagtttccaaccttctccccaacccttggagctaaatgcgttgtaaaatattgccaaaatgaaaagtgttttgtaatactgcaataaaggctgcttgtttttgtggaaaaaaaaaaaaaaaaaaaaaa

SEQ ID NO: 25 - DNA, 1410 bp, Homo sapiens
gtcctcccgccccgccgcttggtggcggccgcatgctgcccggatataaagggtcggccccacatcccagggaccagcgagcggccttgagaggctctggctcttgcttcttaggcggcccgaggacgccatggccgagtgcccgacactcggggaggcagtcaccgaccacccggaccgcctgtgggcctgggagaagttcgtgtatttggacgagaagcagcacgcctggctgcccttaaccatcgagataaaggataggttacagttacgggtgctcttgcgtcgggaagacgtcgtcctggggaggcctatgacccccacccagataggcccaagcctgctgcctatcatgtggcagctctaccctgatggacgataccgatcctcagactccagtttctggcgcttagtgtaccacatcaagattgacggcgtggaggacatgcttctcgagctgctgccagatgactgatgtatggtcttggcagcacctgtctcctttcaccccagggcctgagcctggccagcctacaatggggatgttgtgtttctgttcaccttcgtttactatgcctgtgtcttctccaccacgctggggtctgggaggaatggacagacagaggatgagctctacccagggcctgcaggacctgcctgtagcccactctgctcgccttagcactaccactcctgccaaggaggattccatttggcagagcttcttccaggtgcccagctatacctgtgcctcggcttttctcagctggatgatggtcttcagcctctttctgtcccttctgtccctcacagcactagtatttcatgttgcacacccactcagctccgtgaacttgtgagaacacagccgattcacctgagcaggacctctgaaaccctggaccagtggtctcacatggtgctacgcctgcatgtaaacacgcctgcaaacgctgcctgccggtaaacacgcctgcaaacgctgcctgcccgtaaacacgcctgcaaacgctgcctgcccacacaggttcacgtgcagctcaaggaaaggcctgaaaggagcccttatctgtgctcaggactcagaagcctctgggtcagtggtccacatcccgggacgcagcaggaggccaggccggcgagccctgtggatgagccctcagaacccttggcttgcccacgtggaaaagggatagaggttgggtttccccccttttatagatggtcacgcacctgggtgttacaaagttgtatgtggcatgaatactttttgtaatgattgattaaatgcaagatagtttatctaacttcgtgcggaatcagcttctatccttgacttagattctggtggagagaagtgagaataggcagcccccaaataaaaaatattcatggaaaaaaaaaaaaaaa

SEQ ID NO: 26 - DNA, 481 bp, Homo sapiens
ttggaagagctcattgagtttatttaaggaatacaaaggggactggaagtgtcactggcatgtatacacacacacacacacacacacacacacacacacttacacacatgcacacatcctaagcaacattgctccagaggttggcatcatctgctcgaggtcttcagatttcagagtcaggtgtgaatcttggggacttgaggagatccaggctggccacctgaggatcacctggtgctccaggtgcccatgcggccccctcctccccaggctgggtctggcccatcgggattatccatgttctcataagagtctgcatcttcctcatgattgggtccaggctggccccgaatggagcggagctggggggctgcatacaggattcctctcatatcctcataggactgggaccccagggaggttgcttcccggcttggatcccaggctgacccatgagggctcaggaaatccattgtcctgg

SEQ ID NO: 27 - DNA, 435 bp, Homo sapiens
  misc_feature (77)..(77): n is a, c, g, or t
ttttgctggtgtctgagcttcagtattaaagacaaaacttcctgtttgtggaacaaaagttcgaaagaaaaaacaanacaaaaacacccagccctgttccaggagaatttcaagttttacaggttgagcttcaagatggtttttttggttttttttttttctctcatccaggctgaaggattttttttttctttacaaaatgagttcctcaaattgaccaatagctgctgctttcatattttggataagggtctgtggtcccggcgtgtgctcacgtgtgtatgcacgtgtgtgtgtccattagacacggctgatgtgtgtgcaaagtatccatgcggagttgatgctttgggaattgggctcatgaaggttcttctcaagggttcgagctcatccccctctctccttcctgcctattgactgggagactgtgct

SEQ ID NO: 28 - DNA, 441 bp, Homo sapiens
  misc_feature (441)..(441): n is a, c, g, or t
tttgaatcatcttttgaatgtgtagatcagaaacaaattgaagaacaaaaggaagaagaaaaaatacgggaacagcaagtgaaagaacgaaggcagagagaagaaagaaggcagagtaacctacaagaagttttagaaagggaacgaagagaactagaaaaacctgtatcaggaaaggaagatgattgaagaatcacctgaagattaaaataaaaaaggaattagaaatggaaaatgaattagaaatgagtaatcaagaaataaaagacaaatctgctcacagtgaaaatcctttagagaaatacattgaaaatcatccagcaggagcaagaccaggagtccggcagataagagctccaaaaagatggtccaagaaggctcccctatgttggacaccgctgccatcctagtgaccaagtcgaaagttttaacaggcttttn

SEQ ID NO: 29 - DNA, 499 bp, Homo sapiens
ccacaaaagcacttttaatttgaggcaaagagaagtcttgctgaaaggattccagttccaagcagtcaaaactcaacggttagtggcactattttgacctggtagattttgcttctctttggtcagaaaagggtatccaggttgtactttccccagcagggtaagaaagaagggcaaagaaaattggaagagaccttcaactctactgacagggcccttgagatccaaccatcaagctagaacacgccttcgctgggcaatctacaggttgctgtcccactgctgagtgacacaggccatactacatttgcaaggaaaaaaatgagggcaagaaacacaggtataggtcacttgggggacgagcaggcaaccacagcttcaaaactcttcatggaaggggtaatccctgtgggaggtacagctcaacaaggcacagaccctccagttcctgttgtagctgagtaaggtggtcatatcctggctgctcagttcaaagtca

SEQ ID NO: 30 - DNA, 385 bp, Homo sapiens
  misc_feature (11)..(11): n is a, c, g, or t
aaatttgtggnttctcatnaatanaccgtttattccacgggggaaggcactggaggccagggctccctggggtgggagtggttgcgggagaggaatgatgttcctgtggctcttctggggcccagttgggtccatggcccttcccaagctctgctgggaggccctcggctccgagtccatttgcgggctctggaccagtctctgcctcccccatcccatgtgtggggacggatcaactcatagcacccccagaaggctgggtcccgtccatccccagagaactccctccaagttgctgccctgttgtccttctactccagggccctttggcaagagctgggacaccagggggntggagacaaatggcagntttgggtggggccag

SEQ ID NO: 31 - DNA, 432 bp, Homo sapiens
  misc_feature (340)..(340): n is a, c, g, or t
ctatctgtgaaaaacatttattctgagaatctaaaatctggacaaagtactggactttagaaaaagcctacacaaaattgtctcattcttccctaatacattaataatctaagaataaggaggtgaaaaaaaccctttaaaaataacattgctccagtttgtctgcaggtatgtgatttaaaatatccctgttttattgaggtataggctgcaaactttggtaaaattacgaaaaattaacaaaccctttcaaaagaaaaaaaatacaaaaaaaaaaacaaccaaaccctaacgtcatatctttcttgggtacatatacagtaactacaaaaaaaattcntttagaaaatgnggttccttaagagaaaaaaaggngaaaaaaaaccccccctnggtaaggtccccacttggccggggtttaaaaaattggna

SEQ ID NO: 32 - DNA, 2910 bp, Homo sapiens
gcccaggtgactgactaatccgccgccttcaggagacagaattggccaaggcctgaaggccccggagtgcaccagcggcatgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacagaccctggctcgcctaagaaatgccgtgctcgctttggcctcaaccagcagacggattggtgtggtccgtgcagataactctcttcactattcctaggaggaaaaagaaatgcattcggtacttacccggagaaggccgctgccccagccccgttccttccgatgacagtgctctaggctgccccgggtccccagctccccaggactcaccctcataccatctgctgccccgcttccccacagaactgcttactagccctgcggagcggcacctacatccccaggtctctccactgctctcagcctcccaaccccagggcccccacaggccccccgcagcaccctgcagagcacacaggtacagcaacaggaatctcagagacaggtggcctagcaggcacaggacacctggccgcctccaggagcctaccccctgaaaatgacagagacccagatctcatggaaactggcaggggtcctgttaacgtcatctcagggtccagaccctgaagatttcagaggctgcagaacttctgcctgaacctggggtcatcgattcaaactgctccaagtggtgggaatcagatctgtcttgatgtgtcatctaattaagggaatcccttgtacctatgactccgtcgatctattctttgtaccatctgtcttgccagccagaagcctctgcctccctagcttttctgctataggtcagagatgggctgaactgagcctagctaccttctctacccatctcccccatcccccactgccacaccctccccattcagacacttcatggaccaagaatgagctggtttgtcaaacaacatgtgagcatggtcacaagcacaaagctcaagatgacagctcttctaaggaaatggagaagctctgtttataaaaacaaaaacaaaaccagctgctactcataagttggaccagaggaagccccttactatgatctcaggagcttgcaagaagcaggaaggggaatggaataggttaagtttaggcctatcaacctaagcaacagaaataatctgacactaccttatcaggcaaattggggaggggagggtgtatctagctctagttcaaattatttggaagtgttccctgagaaacccaccagcctaagaagctctggccccaggcttgtcactagcagctgcagtcaacagttcaaagaagtcatggcccaaatccagtgtgcacccctccccattcacagagcctttttcacaattccatttccagttcatctatggcagtccagccagcttctgggcagcttgagagggcaaacccaaaacctcatgacagccagagcctgtctttcagcattcagtcgcctgggaagctccagtttccccatggggctgcgggacagaggaccattacaactagatcaaggagcccagaaaacctccagtagtggacaacaggttttcaccatagcctacgttaacccatttttgagccaagcttcaaccctcagccttgaaaaacaagtctttaatttaatttttgttttttgcctaaatccaaagaaaaagggctgccgggccaggcgcggtcgtcacgcctgtaatcccagcactttggcaggccgaggcaggtggatcacctgacgtcagtagtttgagaccagcctggccaacatggtgaaaccctgtatctactaaaaatacaaaaattagccggacgtggtggtgcgcgcatgtaatcccagctactcgggaggctgaggcggaagaatcacttgaacccgggaggcggaggttccagtgagccgagatggcgctattgcactccagtctgggtaacagggagactgcatctcaaaaaaaaaaaaaaaaaaaaagggctgtccatgtattcacacacccctcaaaaaaagcctttagttcctactttagccactggtttctcagaatccaaagatcacatattctagtgtaacactgcaagaagtcttgagaaaaagattattgtagtgttcaaaatatttttgtattgttaatgcatcatcatagaaaaacttttaaacatgagaataaagatactttttactgggtttgtttttcaaagcctgaaaaaaa

SEQ ID NO: 33 - DNA, 1151 bp, Homo sapiens
ctggtctagaatattccacatctgctctcactctgccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttctcgacctgttcggctaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaaggcagccctggaagtggagttaggagcttctaacccgtcatggttcaatacacattcttcttttgccagcgcttctgaagagctgctctcacctctctgcatcccaatagatatccccctatgtgcatgcacacctgcacactcacggctgaaatctccctaacccagggggac

SEQ ID NO: 34 - DNA, 1586 bp, Homo sapiens
gaatcaggaagaccagctcctcctactgtcttctgtgttacgggatcagcgttccttgttgagtgggacctgagttttgagagggtcttctgctcctcttggtctggtcccttacttccaagagccccagagaggaaggcatgctgttggctctagctctgcttctagctttcctgcctcctgccagtcagaaatcttccaacttggaagggagaacaaagtcagtcaccaggccaactgggtcatcagctgtaatcacttgtgatcttcctgtagaaaatgccgtctacacccactggtacctacaccaggaggggaaggccccacagcgtcttctgtactatgactcctacaactccagggttgtgttggaatcaggaatcagtcgagaaaagtatcatacttatgcaagcacagggaagagccttaaatttatactggaaaatctaattgaacgtgactctggggtctattactgtgccacctggaaggattattataagaaactctttggcagtggaacaacacttgttgtcacagataaacaacttgatgcagatgtttcccccaagcccactatttttcttccttcgattgctgaaacaaaactccagaaggctggaacatatctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaagacagttattcaaaagatgcaaatgatgtcaccacagtggatcccaaatacaattattcaaaggatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaataagcaaaataaattt

SEQ ID NO: 35 - DNA, 896 bp, Homo sapiens
ggggaaagcagcccattcctgggtcagagactcccaccccagctcagagggagcaggggcccagcagggacggaccctcattcctcccagggaccccagacctctgtctctctcgggccttggctccttgaacttttggccgccatgtgcttcccgaagtcctctctgatgacatgaagaagctgaaggcccgaatgcaccaggccatagaaagattttatgataaaatgcgaaatgcagaatcaggacgtggacaggtgatgtcgagcctggcagagctggaggacgacttcaaagagggctacctggagacagtggcggcttattatgaggagcagcacccagagctcactcctctacttgaaaaagaaagagatggattacggtgccgaggcaacagatcccctgtcccggatgttgaggatcccgcaaccgaggagcctggggagagcttttgtgataaggtcatgaggtggttccaggccatgctgcgacggctgtagacctggtggcacggggtcctggcctgggtgaaggagaaggtggtggccctggtccatgcagtgcaggccctctggaaacagttccagagtttctgctgctctctgtcagagctcttcatgtcctctttccagtctacggagcacggggggacaaggaggagctgacaccccagaagtgttctgaaccccaatcctcaaaatgaagatactgacaccacctttgccctccccgtcaccgcgcacccaccctgacccctccctcagctgtcctgtgccccaccctctcccgcacactcagtccccctgcctgggcgttcctgccgcagctctgacctggcggtcgccctggcatcttaataaaacctgcttatacttcctggcaggggagatacc

SEQ ID NO: 36 - DNA, 3030 bp, Homo sapiens
ctgcgaggagtcacattaactttgctctagaagacaactttacaaggatctaaaaggaacaggattaaagatgactgaatactgggttccagaaatttaaaacaatcagcttagcaaatcatatattcttctgtggagctgagaattgatgtccgctcttccccgtgatttggaactttccaatcccagagaaaagttgacaaagggactgcccaggactgagtccatatggaagaagaacttcctcttttctctggagacagtggcaagccagtacaggctactctgtcatctttgaagatgttagatgtgggaaagtggccaattttttccctttgttctgaagaagaactacagttaattcgtcaggcttgtgtctttggcagtgctggcaatgaagttttatacactacagtaaatgatgagatttttgtgcttggcacaaactgctgtggctgtttggggttaggtgacgtccagagcaccattgaacctcggagactggattctttaaatggcaaaaaaatagcctgcctcagctatgggagtggtccacatattgtccttgcaacaacagaaggagaagtctttacctggggtcataatgcttatagccagctgggcaatgggacaactaatcatggtttagtgccctgtcatatctctactaatctgtcaaacaaacaagtcattgaagttgcctgtgggtcttaccattctttggtgctaacatctgatggagaggtatttgcctggggttataataactctgggcaggtaggatctggatcaacagttaatcagccaatccctcgaagagtcactggctgcctacaaaataaagtagttgtgaccatagcatgtgggcagatgtgctgcatggcagtagtagacacgggggaggtctatgtctggggttacaacggaaacgggcagcttggactcggcaacagtggcaaccagccaaccccttgcagagtggcagctttgcaaggcatccgtgtccagagggtcgcctgtggctacgcacacacattagtattaacagatgaaggccaagtgtatgcttggggcgccaattcttatgggcagttgggcactggcaataaaagcaaccagtcctatcctactcctgtcactgtggaaaaggacaggattatcgagattgcagcctgtcactccacacacacgtctgcggccaagacgcagggtgggcacgtgtacatgtggggccagtgccggggtcagtccgtgatcctcccgcacctcacccacttctcctgcactgacgacgtgtttgcctgctttgccacgcccgccgtcacgtggcgcctcctctccgtggaacctgatgaccacctcacagtggctgagtcactgaagagggaatttgacaacccggacactgcagacctgaagtttctagttgatggaaagtacatttatgcacataaagtccttctcaagattcgatgtgagcattttcgttcgtcattggaagataacgaggatgatattgtagaaatgagtgaattttcatatcctgtttaccgggccttcctggaatacctatacacagacagcatcagcctttctcctgaggaggcagtaggactgctagacttggctacattttatagagaaaatcgtttgaaaaagctctgccaacaaactatcaagcaaggcatctgcgaggagaatgccatcgctctgctctcggctgcggtgaagtatgatgcacaggatttagaagaattctgcttcaggttttgcataaaccatctgactgtagtaacacaaacatcaggttttgcagaaatggaccatgatctcctgaagaactttatcagcaaagcaagcagagttggagcctttaaaaattgatcccatctgcaggaaagtttttgagcctttccatttcccctgcaaaagccagagatgaatcacttctctttaattaatagtatgtatgatgagctatgtttggctgagtacttgtaactgtcagaagaaggatggtggtgagtggtctttgtctgcctaaacccagagtttatgtagaaagcattgaatgttctgatcagatgtgactaaggtcaaggaaaaaaaattgaaatatcttatttaccatttcctctttttgagtcacttaaattggacacctttggtaccctggtctcagtatatgctattctggcccaaatgttccattattcagctggctgataccacatagatagcttgacaaggagtgctgtctgtccttaccacattttcagcactcagcacagtgccttgtgtataataggcactcaatttattataaatcttcagtatgtcctgagaacagctttagtcatggaatactgggagaaggaataactttcacaaaataaacttaaaacagcctgtaattattgaggttcacattcttctggtatatcattctgagaaattgtggctaatttagaacattgtttagaattgacaaaaggccctggcaattaaattgtcaaggcccaagggctaattttaattttctttttacttggagtcattcattaatttctcacatgggattatggagtatgaagtattatctttgaatgaaattcctgggctgatctgccttacataatcacataaggtcctttgcttttctttgtgttaagagggacttgcctctgtaaatgaaaatgacaatgtgcttttcttgtagttgactttcatgtcactcactataaaataggtctcttaacctggcaccagtataactataaagcactagctgagaaggaactgatacttacatttcatggacagcattaacaagaatgagataaatttgtactttttagatcaaaacaaattacctaattgcaaaagagaaactgaaatggaacatagtctcagattcttctaatgtgtatctcacaatgtcatgtaatgtaaaggaaacccttttggaattagaattcttgttctgatgctgaactatttggtaataaagtgcttatttgcagataacag

(19) United States
(12) Patent Application Publication
(10) Pub. No.: US 2012/0015845 A1
(43) Pub. Date: Jan. 19, 2012

(54) MARKERS FOR THE DIAGNOSIS OF AML, B-ALL AND T-ALL

(76) Inventors: Jeong Ho Yoon, Seoul (KR); Se Nyun Kim, Seoul (KR); Young-Hwa Song, Paju-si (KR); Dong Yoon Park, Seoul (KR); Sung Han Kim, Bucheon-si (KR); InKyung Shin, Yongin-si (KR); Yeo Wook Koh, Seongnam-si (KR)

(21) Appl. No.: 13/241,085

(22) Filed: Sep. 22, 2011

Related U.S. Application Data
(62) Division of application No. 11/772,048, filed on Jun. 29, 2007.
Application No. 11/772,048 is a continuation of application No. PCT/KR05/04630, filed on Dec. 29, 2005.

(30) Foreign Application Priority Data
Dec. 29, 2004 (KR) 10-2004-0115526

(51) Int. Cl. C40B 30/04; G01N 33/574; C12Q 1/68

(52) U.S. Cl. 506/9; 435/6.12; 435/6.14; 435/7.92; 436/501; 435/7.1; 435/7.4; 435/7.23

(57) ABSTRACT

Disclosed are diagnostic markers specific for acute myeloid leukemia (AML), B-cell lineage acute lymphoblastic leukemia (B-ALL), and T-cell lineage acute lymphoblastic leukemia (T-ALL). Also disclosed are a composition and a kit, comprising an agent detecting the presence of the markers, and a method of diagnosing AML, B-ALL and T-ALL using the same.

### CROSS-REFERENCE TO RELATED APPLICATIONS

The present application is a divisional of U.S. patent application Ser. No. 11/772,048, filed Jun. 29, 2007 (pending), which claims the benefit of priority to Republic of Korea Patent Application Serial No. 10-2004-0115526, filed Dec. 29, 2004, the contents of which are incorporated by reference herein in their entirety.

### TECHNICAL FIELD

The present invention relates to diagnostic markers specific to acute myeloid leukemia (AML), B-cell acute lymphoblastic leukemia (B-ALL), and T-cell acute lymphoblastic leukemia (T-ALL). More particularly, the present invention relates to compositions and kits comprising agents detecting the presence of the markers, and methods of diagnosing AML, B-ALL and T-ALL.

### BACKGROUND ART

Leukemia is a group of diseases characterized by the malignant proliferation of white blood cells (leukocytes). Leukemia is divided into myelogenous and lymphocytic types based on its origin, and into acute and chronic types based on how quickly it progresses. The clinical symptoms of leukemia vary depending on the disease type and the nature of involved cells. When leukemia affects lymphoid cells, it is called lymphocytic leukemia (also known as lymphoid or lymphoblastic leukemia). When myeloid cells are affected, the disease is called myeloid leukemia (also known as myelogenous or myelocytic leukemia). Chronic myeloid leukemia is caused by the abnormal growth of myeloid cells. Acute myeloid leukemia results from the aberrant differentiation and proliferation of myeloid progenitor cells that begin to differentiate at relatively early stages of hematopoiesis.

The different types of acute leukemia are caused by different mechanisms and treated with different therapeutic agents or different therapies. Thus, it is very important to accurately diagnose the type of leukemia. Acute leukemia has been diagnosed primarily by observing bone marrow cells under a microscope to examine the morphology and staining patterns of abnormal cancer cells. Also, an immunological method that uses monoclonal antibodies to proteins in order to help in the diagnosis is used. However, there has still been no general approach for pathologically or histologically distinguishing the different types of acute leukemia.

In recent years, microarray techniques, which can screen the expression of several thousands to several thousand tens of genes at one time, have been developed. Golub et al. reported that AML and ALL can be distinguished based on monitoring the expression of fifty genes (Golub et al., Science 1998, 286:531-537). This report shows the possibility of diagnosing AML and ALL by monitoring gene expression, but this method has not been assessed for the practical applicability and thus has limited applicability in practical diagnosis.

Thus, there is a need for the development of highly significant markers capable of rapidly and accurately distinguishing AML and ALL.

In this regard, to develop biological markers capable of simply and accurately distinguishing AML, B-ALL and T-ALL, the present inventors performed primary screening to identify genes overexpressed only in each type of leukemia using a DNA chip, and selected highly significant markers using RT-PCR. As a result, the present inventors identified genes useful as potential markers for AML, CITED2, MGST1, BIN2, RAB32, ICAM-3, PXN, PPGB and TAF15; genes useful as potential markers for B-ALL, TCL1A, CD19, INSR, OFD1, AKR1B1, CD79B and UHRF1; and genes useful as potential markers for T-ALL, TCF7, TRB, TRGC2, NK4 and CHC1L. When these potential markers were applied in practice to leukemia samples, they were found to rapidly, simply and accurately diagnose the different types of leukemia, thereby leading to the present invention.

### DISCLOSURE OF THE INVENTION

It is therefore an object of the present invention to provide a kit for detecting a diagnostic marker for AML, comprising an agent measuring mRNA or protein levels of (i) the CITED2 gene, or (ii) the CITED2 gene and one or more genes selected from among MGST1, BIN2, RAB32, ICAM-3, PXN, PPGB and TAF15.

It is another object of the present invention to provide a kit for detecting a diagnostic marker for B-ALL, comprising an agent measuring mRNA or protein levels of (i) the TCL1A gene, or (ii) the TCL1A gene and one or more genes selected from among CD19, INSR, OFD1, AKR1B1, CD79B and UHRF1.

It is a further object of the present invention to provide a kit for detecting a diagnostic marker for distinguishing between AML, B-ALL and T-ALL, comprising an agent measuring mRNA or protein levels of (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L.

It is yet another object of the present invention to provide a composition for detecting a diagnostic marker for AML, comprising a pair of primers specific to (i) the CITED2 gene, or (ii) the CITED2 gene and one or more genes selected from among MGST1, BIN2, RAB32, ICAM-3, PXN, PPGB and TAF15.

It is still another object of the present invention to provide a composition for detecting a diagnostic marker for AML, comprising an antibody specific to (i) the CITED2 protein, or (ii) the CITED2 protein and one or more proteins selected from among MGST1, BIN2, RAB32, ICAM-3, PXN, PPGB and TAF15.

It is still another object of the present invention to provide a composition for detecting a diagnostic marker for B-ALL, comprising a pair of primers specific to (i) the TCL1A gene, or (ii) the TCL1A gene and one or more genes selected from among CD19, INSR, OFD1, AKR1B1, CD79B and UHRF1.

It is still another object of the present invention to provide a composition for detecting a diagnostic marker for B-ALL, comprising an antibody specific to (i) the TCL1A protein, or (ii) the TCL1A protein and one or more proteins selected from among CD19, INSR, OFD1, AKR1B1, CD79B and UHRF1.

It is still another object of the present invention to provide a composition for detecting a diagnostic marker for distinguishing between AML, B-ALL and T-ALL, comprising a pair of primers specific to (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L.

It is still another object of the present invention to provide a composition for detecting a diagnostic marker for distinguishing between AML, B-ALL and T-ALL, comprising an antibody specific to (a) (i) the CITED2 protein, or (ii) the CITED2 protein and one or more proteins selected from among MGST1, BIN2, RAB32, ICAM-3, PXN, PPGB and TAF15; (b) (i) the TCL1A protein, or (ii) the TCL1A protein and one or more proteins selected from among CD19, INSR, OFD1, AKR1B1, CD79B and UHRF1; and (c) (i) the TCF7 protein, or (ii) the TCF7 protein and one or more proteins selected from among TRB, TRGC2, NK4 and CHC1L.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows the results of RT-PCR for detecting the expression of two genes specifically expressed in each of B-ALL (B-cell acute lympnoblastic leukemia), T-ALL (T-cell acute lympnoblastic leukemia) and AML (acute myeloid leukemia), among genes listed in Tables 1, 2 and 3, and two control genes expressed at constant levels in all leukemia cells, in four ALL specimens and four AML specimens;

FIG. 2 shows the results of RT-PCR for detecting the expression of eight diagnostic marker genes in AML patients having normal chromosomes, wherein all patients except patient 21 expressed only AML marker genes and control genes;

FIG. 3 shows the results of RT-PCR for detecting the expression of eight diagnostic marker genes in AML patients having a t(15;17) chromosomal abnormality, wherein all patients expressed only AML marker genes and control genes;

FIG. 4 shows the results of RT-PCR for detecting the expression of eight diagnostic marker genes in AML patients having a t(8;21) chromosomal abnormality, wherein all patients expressed only AML marker genes and control genes;

FIG. 5 shows the results of RT-PCR for detecting the expression of eight diagnostic marker genes in B-ALL patients having normal chromosomes, wherein all patients expressed only B-ALL marker genes and control genes;

FIG. 6 shows the results of RT-PCR for detecting the expression of eight diagnostic marker genes in B-ALL patients having a t(9;22) chromosomal abnormality, wherein all patients except patient 51 expressed only B-ALL marker genes and control genes; and

FIG. 7 shows the results of RT-PCR for detecting the expression of eight diagnostic marker genes in T-ALL patients, wherein all patients expressed only T-ALL marker genes and control genes.

### BEST MODE FOR CARRYING OUT THE INVENTION

The present invention relates to markers for diagnosing acute leukemia, which is characterized in that white blood cells reproduce without undergoing normal differentiation, causing immature, abnormal white blood cells to accumulate in the bone marrow and peripheral blood, and preferably acute myeloid leukemia (AML), B-cell acute lymphoblastic leukemia (B-ALL) and T-cell acute lymphoblastic leukemia (T-ALL). The present invention provides a method of easily and simply diagnosing each type of acute leukemia by detecting a diagnostic marker specific to each type of acute leukemia in a biological sample.

The terms “markers for diagnosing”, “markers for diagnosis”, or “diagnostic markers”, as used herein, are intended to indicate substances that can diagnose leukemia by distinguishing leukemia cells from normal cells, and includes organic biological molecules, quantities of which increase or decrease in leukemia cells compared to normal cells, such as polypeptides or nucleic acids (e.g., mRNA, etc.), lipids, glycolipids, glycoproteins, and sugars (monosaccharides, disaccharides, oligosaccharides, etc.). These markers, associated with specific conditions, phenotypes or cell types, can be detected through analysis. With respect to the objects of the present invention, leukemia diagnostic markers are nucleic acid and polypeptide markers capable of diagnosing AML, B-ALL and T-ALL, which have increased expression only in AML cells, B-ALL cells and T-ALL cells, respectively.

The selection and application of significant diagnostic markers are factors that determine the reliability of diagnosis results. A “significant diagnostic marker” means a marker that is highly valid by making an accurate diagnosis and is highly reliable by providing constant results upon repeated measurement. The leukemia diagnostic markers of the present invention, which are genes whose expression always increases due to direct or indirect factors when a specific type of leukemia develops, display the same results upon repeated tests, and have high reliability due to a great difference in expression levels compared to a control and other types of leukemia, thus having a very low possibility of giving false results. Therefore, a diagnosis of leukemia type based on the results obtained by measuring the expression levels of the significant diagnostic markers of the present invention is valid and reliable.

The term “biological sample”, as used herein, refers to tissues, cells and others, in which a difference in expression levels of a gene or protein used as a leukemia diagnostic marker can be detected when leukemia develops. Examples of the biological samples include, but are not limited to, bone marrow, lymph nodes, spleen, peripheral blood, lymph fluid, serous fluid, urine, and saliva.

The term “diagnosis”, as used herein, refers to the identification of the presence or properties of pathological states. With respect to the objects of the present invention, the diagnosis indicates the identification of the incidence of acute leukemia, and is characterized by accurately identifying the type of acute leukemia, which is classified into AML, B-ALL and T-ALL.

In one aspect, the present invention relates to a method of diagnosing acute myeloid leukemia (AML), comprising measuring mRNA or protein levels of (i) the CITED2 gene or (ii) the CITED2 gene and one or more genes selected from among MGST1, BIN2, RAB32, ICAM-3, PXN, PPGB and TAF15 in a biological sample from a patient suspected of having leukemia; and comparing mRNA or protein levels of the sample from the patient with those of a normal control sample to determine the increase in mRNA or protein levels.

The term “acute myeloid leukemia (AML)”, as used herein, refers to a malignant blood disease in which abnormally differentiated myeloid cells reproduce in the bone marrow and spread to the peripheral blood or other organs. This disease most often occurs in adults.

The genes of the present invention, CITED2 (Cbp/p300-interacting transactivator with Glu/Asp-rich carboxy-terminal domain, 2), MGST1 (microsomal glutathione S-transferase 1), RAB32 (RAB32, a member of the RAS oncogene family), BIN2(bridging integrator 2), ICAM-3 (Intercellular Adhesion Molecule-3), PXN (Paxillin), PPGB (protective protein for beta-galactosidase) and TAF15 (TAF15 RNA polymerase II, TATA box binding protein (TBP)-associated factor, 68 kDa), are expressed in high levels specifically in AML cells relative to normal cells and other types of acute leukemia cells, and thus are provided as diagnostic markers for AML.

Herein, RRAGB (GTP-binding protein raga) and/or DCK (deoxycytidine kinase) genes, which are expressed in almost the same levels in all types of acute leukemia, are used as quantitative controls.

The CITED2 gene is scarcely expressed in B-ALL and T-ALL, but is expressed specifically in AML. Thus, this gene is a highly reliable marker which allows the sensitive, accurate and highly precise diagnosis of AML even when used alone. Therefore, the diagnosis of AML is carried out by detecting the CITED2 marker gene alone, or by detecting markers which are essentially composed of the CITED2 gene along with one or more genes selected from among MGST1, BIN2, RAB32, ICAM-3, PXN, PPGB and TAF15. Preferably, the MGST1 gene is selected and used as an AML diagnostic marker in combination with the CITED2 gene.

Expression levels of genes in biological samples may be determined by measuring mRNA or protein levels. The mRNA or protein isolation from a biological sample may be carried out using a known process (Chomczynski and Sacchi Anal. Biochemistry. 1987, 162: 156-159).

The term “the measurement of mRNA expression levels”, as used herein, refers to a process of assessing the presence and expression levels of mRNA of AML marker genes in biological samples for diagnosing AML, in which the amount of mRNA is measured. Analysis methods for measuring mRNA levels include, but are not limited to, RT-PCR, competitive RT-PCR, real-time RT-PCR, RNase protection assay (RPA), Northern blotting, and DNA chip assay.

With the detection methods, a patient suspected of having AML may be compared with a normal control for mRNA expression levels of an AML marker gene, and the patient's suspected AML may be diagnosed by determining whether expression levels of mRNA from the AML marker gene have significantly increased.

The measurement of mRNA expression levels is preferably carried out by RT-PCR using primers specific to a gene used as an AML diagnostic marker.

RT-PCR is a method that was introduced by P. Seeburg to analyze RNA (Cold Spring Harb Symp Quant Biol 1986, Pt 1:669-677), with which cDNA is synthesized from mRNA using reverse transcription, amplified by PCR, and analyzed. At the amplification step, a pair of primers prepared in a manner as to be specific to an AML diagnostic marker is used. RT-PCR products are electrophoresed, and patterns and thicknesses of bands are analyzed to determine the expression and levels of mRNA from a gene used as an AML diagnostic marker while comparing the mRNA expression and levels with those of a control, thereby simply diagnosing the incidence of AML.

Alternatively, the measurement of mRNA expression levels is carried out using a DNA chip in which the AML marker genes or nucleic acid fragments thereof are anchored at high density to a glass-like base plate. A cDNA probe labeled with a fluorescent substance at its end or internal region is prepared using mRNA isolated from a sample, and is hybridized with the DNA chip. The DNA chip is then read to determine the presence or expression levels of the genes, thereby diagnosing the incidence of AML.

The term “the measurement of protein expression levels”, as used herein, is a process of assessing the presence and expression levels of proteins expressed from AML marker genes in biological samples for diagnosing AML, in which the amount of protein products of the marker genes is measured using antibodies specifically binding to the proteins.

Analysis methods for measuring protein levels using antibodies include, but are not limited to, Western blotting, enzyme linked immunosorbent assay (ELISA), radioimmunoassay (RIA), radioimmunodiffusion, ouchterlony immunodiffusion, rocket immunoelectrophoresis, immunohistostaining, immunoprecipitation assay, complement fixation assay, FACS, and protein chip assay.

With the analysis methods, a patient suspected of having AML is compared with a normal control for the amount of formed antigen-antibody complexes, and the patient's suspected AML is diagnosed by evaluating a significant increase in expression levels of a protein from the AML marker gene.

The term “antigen-antibody complexes”, as used herein, refers to binding products of an AML marker protein to an antibody specific thereto. The amount of formed antigen-antibody complexes may be quantitatively determined by measuring the signal size of a detection label.

Such a detection label may be selected from the group consisting of enzymes, fluorescent substances, ligands, luminescent substances, microparticles, redox molecules and radioactive isotopes, but the present invention is not limited to the examples. Examples of enzymes available as detection labels include, but are not limited to, β-glucuronidase, β-D-glucosidase, β-D-galactosidase, urase, peroxidase or alkaline phosphatase, acetylcholinesterase, glucose oxidase, hexokinase and GDPase, RNase, glucose oxidase and luciferase, phosphofructokinase, phosphoenolpyruvate carboxylase, aspartate aminotransferase, phosphenolpyruvate decarboxylase, and β-latamase. Examples of the fluorescent substances include, but are not limited to, fluorescin, isothiocyanate, rhodamine, phycoerythrin, phycocyanin, allophycocyanin, o-phthaldehyde and fluorescamin. Examples of the ligands include, but are not limited to, biotin derivatives. Examples of luminescent substances include, but are not limited to, acridinium esters, luciferin and luciferase. Examples of the microparticles include, but are not limited to, colloidal gold and colored latex. Examples of the redox molecules include, but are not limited to, ferrocene, ruthenium complexes, viologen, quinone, Ti ions, Cs ions, diimide, 1,4-benzoquinone, hydroquinone, K₄W(CN)₈, [Os(bpy)₃]²⁺, [RU(bpy)₃]²⁺, and [MO(CN)₈]⁴⁻. Examples of the radioactive isotopes include, but are not limited to, ³H, ¹⁴C, ³²P, ³⁵S, ³⁶Cl, ⁵¹Cr, ⁵⁷CO, ⁵⁸CO, ⁵⁹Fe, ⁹⁰Y, ¹²⁵I, ¹³¹I, and ¹⁸⁶Re.

Preferably, the measurement of the protein expression levels is carried out by ELISA. Examples of ELISA include direct ELISA using a labeled antibody recognizing an antigen immobilized on a solid support; indirect ELISA using a labeled antibody recognizing a capture antibody forming complexes with an antigen immobilized on a solid support; direct sandwich ELISA using a labeled antibody recognizing an antigen bound to a antibody immobilized on a solid support; and indirect sandwich ELISA, in which a captured antigen bound to an antibody immobilized on a solid support is detected by first adding an antigen-specific antibody, and then a secondary labeled antibody which binds to the antigen-specific antibody. More preferably, the protein expression levels are detected by sandwich ELISA, where a sample reacts with an antibody immobilized on a solid support, and the resulting antigen-antibody complexes are detected by adding a labeled antibody specific for the antigen, followed by enzymatic development, or by adding first an antigen-specific antibody and then a secondary labeled antibody which binds to the antigen-specific antibody, followed by enzymatic development. The incidence of AML may be diagnosed by measuring the degree to which an AML marker protein and an antibody thereto form complexes.

In addition, the measurement of the protein expression levels is preferably carried out using a protein chip in which antibodies to the AML markers are arrayed and immobilized at predetermined positions of a base plate at high density. Using a method of analyzing a sample using a protein chip, proteins are isolated from the sample and hybridized with the protein chip to form antigen-antibody complexes. The protein chip is then read to determine the presence or expression levels of the proteins, thereby diagnosing the incidence of AML.

Further, the measurement of protein expression levels is preferably achieved using Western blotting using antibodies to the AML makers. Total proteins are isolated from a sample, electrophoresed to separate them according to size, transferred onto a nitrocellulose membrane, and reacted with an antibody. The amount of proteins produced by gene expression is determined by measuring the amount of antigen-antibody complexes produced using a labeled antibody, thereby diagnosing the incidence of AML.

The detection methods comprise methods of assessing the expression levels of maker genes both in a control not having leukemia and in cells in which leukemia occurs. mRNA or protein levels may be expressed as an absolute (e.g., μg/ml) or relative (e.g., relative intensity of signals) difference in the amount of marker proteins.

In another aspect, the present invention relates to a method of diagnosing B-cell acute lymphoblastic leukemia (B-ALL), comprising measuring mRNA or protein levels of (i) the TCL1A gene, or (ii) the TCL1A gene and one or more genes selected from among CD19, INSR, OFD1, AKR1B1, CD79B and UHRF1 in a biological sample from a patient suspected of having leukemia; and comparing mRNA or protein levels of the sample from the patient with those of a normal control sample to determine the increase in mRNA or protein levels.

The term “B-cell acute lymphoblastic leukemia (B-ALL)”, as used herein, includes B-ALL with chromosomal abnormalities as defined by the World Health Organization (WHO) classification, the chromosomal abnormalities including t(8;14), t(8;22), t(2;8), t(9;22), t(4;11) and t(1;19).

The genes of the present invention, TCL1A (T-cell leukemia/lymphoma 1A), CD19, INSR (insulin receptor), OFD1 (oral-facial-digital syndrom 1), AKR1B1 (aldo-keto reductase family 1, member B1), CD79B and UHRF1 (ubiquitin-like, containing PHD and RING finger domains, 1), are expressed at high levels specifically in B-ALL cells relative to normal cells and other types of acute leukemia cells, and thus are used as B-ALL markers.

Herein, RRAGB and/or DCK genes, which are expressed in almost the same levels in all types of acute leukemia, are used as quantitative controls.

The TCL1A gene is rarely expressed in AML and T-ALL, but is expressed specifically in B-ALL. Thus, this gene is a highly reliable marker which enables the sensitive, accurate and highly precise diagnosis of B-ALL even when used alone. Therefore, the diagnosis of B-ALL is carried out by detecting the TCL1A marker gene alone, or by detecting markers which are essentially composed of the TCL1A gene along with one or more genes selected from among CD19, INSR, OFD1, AKR1B1, CD79B and UHRF1. Preferably, the CD19 gene is selected and used as a B-ALL diagnostic marker in combination with the TCL1A gene.

The mRNA levels of the B-ALL markers may be measured using analysis methods that include RT-PCR, competitive RT-PCR, real-time RT-PCR, RNase protection assay (RPA), Northern blotting, and DNA chip assay. With the analysis methods, a patient suspected of having B-ALL may be compared with a normal control for mRNA expression levels of a B-ALL marker gene, and the patient's suspected B-ALL may be diagnosed by determining whether expression levels of mRNA from the B-ALL marker gene have significantly increased. A preferred method is RT-PCR or DNA chip assay, which employs primers specific to a gene used as a B-ALL diagnostic marker.

The protein levels of the B-ALL markers may be measured using analysis methods that include Western blotting, ELISA, RIA, radioimmunodiffusion, ouchterlony immunodiffusion, rocket immunoelectrophoresis, immunohistostaining, immunoprecipitation assay, complement fixation assay, FACS, and protein chip assay. With the analysis methods, a patient suspected of having B-ALL may be compared with a normal control with respect to the amount of antigen-antibody complexes formed, and the patient's suspected B-ALL is diagnosed by evaluating a significant increase in expression levels of a protein from the B-ALL marker gene. A preferred method is Western blotting, ELISA or protein chip assay.

In a further aspect, the present invention relates to a method of diagnosing T-cell acute lymphoblastic leukemia (T-ALL), comprising measuring mRNA or protein levels of (i) the TCF7 gene, or (ii) the TCF7 gene and one or more genes selected from among TRB, TRGC2, NK4 and CHC1L in a biological sample from a patient suspected of having leukemia; and comparing mRNA or protein levels of the sample from the patient with those of a normal control sample to determine the increase in mRNA or protein levels.

The term “T-cell acute lymphoblastic leukemia (T-ALL)”, as used herein, includes T-ALL with chromosomal abnormalities as defined by the WHO classification, the chromosomal abnormalities including 14q11 and 7q34.

The genes of the present invention, TCF7 (transcription factor 7: T-cell specific, HMG-box), TRB (T cell receptor beta locus), TRGC2 (T cell receptor gamma constant 2), NK4 (natural killer cell transcript 4), and CHC1L (chromosome condensation 1-like), are expressed in high levels specifically in T-ALL cells relative to normal cells and other types of acute leukemia cells, and thus are used as T-ALL markers.

Herein, RRAGB and/or DCK genes, which are expressed at almost the same levels in all types of acute leukemia, are used as quantitative controls.

The TCF7 gene is rarely expressed in AML and B-ALL, but is expressed specifically in T-ALL. Thus, this gene is a highly reliable marker which allows the sensitive, accurate and highly precise diagnosis of T-ALL even when used alone. Therefore, the diagnosis of T-ALL is carried out by detecting the TCF7 marker alone, or by detecting markers which are essentially composed of the TCF7 gene along with one or more genes selected from among TRB, TRGC2, NK4 and CHC1L. Preferably, the TRB gene is selected and used as a T-ALL diagnostic marker in combination with the TCF7 gene.

The mRNA levels of the T-ALL markers may be measured using analysis methods that include RT-PCR, competitive RT-PCR, real-time RT-PCR, RNase protection assay (RPA), Northern blotting, and DNA chip assay. With the analysis methods, a patient suspected of having T-ALL may be compared with a normal control for mRNA expression levels of a T-ALL marker gene, and the patient's suspected T-ALL may be diagnosed by determining whether expression levels of mRNA from the T-ALL marker gene have significantly increased. A preferred method is RT-PCR or DNA chip assay, which employs primers specific to a gene used as a T-ALL diagnostic marker.

The protein levels of the T-ALL markers may be measured using analysis methods, which include Western blotting, ELISA, RIA, radioimmunodiffusion, ouchterlony immunodiffusion, rocket immunoelectrophoresis, immunohistostaining, immunoprecipitation assay, complement fixation assay, FACS, and protein chip assay. With the analysis methods, a patient suspected of having T-ALL may be compared with a normal control with respect to the amount of antigen-antibody complexes formed, and the patient's suspected T-ALL is diagnosed by evaluating a significant increase in expression levels of a protein from the T-ALL marker gene. A preferred method is Western blotting, ELISA or protein chip assay.

In yet another aspect, the present invention relates to a diagnosis method for distinguishing between AML, B-ALL and T-ALL, comprising measuring mRNA or protein levels of (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L; and comparing mRNA or protein levels of the sample from the patient with those of a normal control sample to determine the increase in mRNA or protein levels.

With this method, the expression levels of the AML, B-ALL and T-ALL marker genes are measured simultaneously in a single sample from a patient. Thus, this method is effective because it can identify the different types of leukemia at one time.

Herein, RRAGB and/or DCK genes, which are expressed at almost the same levels in all types of acute leukemia, are used as quantitative controls.

More preferably, a method of measuring mRNA or protein levels of CITED2 and MGST1 genes, TCL1A and CD19 genes, and TCF7 and TRB genes is provided.

CITED2 and MGST1 genes are highly significant markers for diagnosing AML. TCL1A and CD19 genes are highly significant markers for diagnosing B-ALL. TCF7 and TRB genes are highly significant markers for diagnosing T-ALL. The incidence and type of leukemia may be diagnosed at one time by comparing the expression patterns and levels of the six genes.

The diagnosis of AML, B-ALL and T-ALL is easily achieved using a kit comprising an agent capable of measuring mRNA or protein levels of the leukemia diagnostic marker genes provided in the present invention.

In still another aspect, the present invention relates to a kit for detecting a diagnostic marker for AML, comprising an agent measuring mRNA or protein levels of (i) the CITED2 gene, or (ii) the CITED2 gene and one or more genes selected from among MGST1, BIN2, RAB32, ICAM-3, PXN, PPGB and TAF15.

The detection kit of the present invention is composed of a composition, solution or apparatus, which includes one or more kinds of different constituents suitable for analysis methods.

Preferably, the present invention relates to a kit for detecting a diagnostic marker, comprising essential elements required for performing RT-PCR. An RT-PCR kit includes a pair of primers specific for each marker gene. The primer is a nucleotide having a sequence specific to a nucleic acid sequence of each marker gene, and is about 7 bp to 50 bp in length, more preferably about 10 bp to 30 bp in length. Also, the RT-PCR kit may include primers specific to a nucleic acid sequence of a control gene. The RT-PCR may further include test tubes or other suitable containers, reaction buffers (varying in pH and magnesium concentrations), deoxynucleotides (dNTPs), enzymes such as Taq-polymerase and reverse transcriptase, DNAse, RNAse inhibitor, DEPC-treated water, and sterile water.

In addition, preferably, the present invention relates to a kit for detecting a diagnostic marker, comprising essential elements required for performing a DNA chip assay. A DNA chip kit may include a base plate, onto which genes or fragments thereof, cDNA, or oligonucleotides are attached, and reagents, agents and enzymes for preparing fluorescent probes. Also, the base plate may include RRAGB and/or DCK genes or fragments thereof, as control genes, such as cDNA.

Further, preferably, the present invention relates to a kit for detecting a diagnostic marker, comprising essential elements required for performing ELISA. An ELISA kit includes antibodies specific to marker proteins. The antibodies are monoclonal, polyclonal or recombinant antibodies, which have high specificity and affinity to each marker protein and rarely have cross-reactivity to other proteins. Also, the ELISA kit may include an antibody specific to a control protein. The ELISA kit may further include reagents capable of detecting bound antibodies, for example, a labeled secondary antibody, chromophores, enzymes (e.g., conjugated with an antibody) and their substrates, or other substances capable of binding to the antibodies.

An RT-PCR kit for detecting AML markers comprises a pair of primers specific to (i) the CITED2 gene, or (ii) the CITED2 gene and one or more genes selected from among MGST1, BIN2, RAB32, ICAM-3, PXN, PPGB and TAF15. Also, the RT-PCR kit may include a pair of primers specific to RRAGB and/or DCK genes.

A DNA chip kit for detecting AML markers includes a base plate onto which cDNA corresponding to (i) the CITED2 gene, or (ii) the CITED2 gene and one or more genes selected from among MGST1, BIN2, RAB32, ICAM-3, PXN, PPGB and TAF15, or fragments thereof, is attached. Also, cDNA corresponding to RRAGB and/or DCK genes, or fragments thereof, may be attached to and immobilized on the base plate.

An ELISA kit for detecting AML markers includes an antibody specific to (i) the CITED2 protein, or (ii) the CITED2 protein and one or more proteins selected from among MGST1, BIN2, RAB32, ICAM-3, PXN, PPGB and TAF15. Also, the ELISA kit may include an antibody specific to RRAGB and/or DCK proteins.

In still another aspect, the present invention relates to a kit for detecting a diagnostic marker for B-ALL, comprising an agent measuring mRNA or protein levels of (i) the CITED2 gene, or (ii) the CITED2 gene and one or more genes selected from among MGST1, BIN2, RAB32, ICAM-3, PXN, PPGB and TAF15.

An RT-PCR kit for detecting B-ALL markers comprises a pair of primers specific to (i) the CITED2 gene, or (ii) the CITED2 gene and one or more genes selected from among MGST1, BIN2, RAB32, ICAM-3, PXN, PPGB and TAF15. Also, the RT-PCR kit may include a pair of primers specific to RRAGB and/or DCK genes.

A DNA chip kit for detecting B-ALL markers includes a base plate onto which cDNA, corresponding to (i) the CITED2 gene, or (ii) the CITED2 gene and one or more genes selected from among MGST1, BIN2, RAB32, ICAM-3, PXN, PPGB and TAF15, or fragments thereof, is attached. Also, cDNA corresponding to RRAGB and/or DCK genes, or fragments thereof, may be attached to and immobilized on the base plate.

An ELISA kit for detecting B-ALL markers includes an antibody specific to (i) the CITED2 protein, or (ii) the CITED2 protein and one or more proteins selected from among MGST1, BIN2, RAB32, ICAM-3, PXN, PPGB and TAF15. Also, the ELISA kit may include an antibody specific to RRAGB and/or DCK proteins.

In still another aspect, the present invention relates to a kit for detecting a diagnostic marker for T-ALL, comprising an agent measuring mRNA or protein levels of (i) the TCF7 gene, or (ii) the TCF7 gene and one or more genes selected from among TRB, TRGC2, NK4 and CHC1L.

An RT-PCR kit for detecting T-ALL markers comprises a pair of primers specific to (i) the TCF7 gene, or (ii) the TCF7 gene and one or more genes selected from among TRB, TRGC2, NK4 and CHC1L. Also, the RT-PCR kit may include a pair of primers specific to the RRAGB and/or DCK genes.

A DNA chip kit for detecting T-ALL markers includes a base plate onto which cDNA corresponding to (i) the TCF7 gene, or (ii) the TCF7 gene and one or more genes selected from among TRB, TRGC2, NK4 and CHC1L, or fragments thereof, is attached. Also, cDNA corresponding to RRAGB and/or DCK genes, or fragments thereof, may be attached to and immobilized on the base plate.

An ELISA kit for detecting T-ALL markers includes an antibody specific to (i) the TCF7 protein, or (ii) the TCF7 protein and one or more proteins selected from among TRB, TRGC2, NK4 and CHC1L. Also, the ELISA kit may include an antibody specific to the RRAGB and/or DCK proteins.

In still another aspect, the present invention relates to a kit for detecting a diagnostic marker for distinguishing between AML, B-ALL and T-ALL, comprising an agent measuring mRNA or protein levels of (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L.

An RT-PCR kit for detecting a diagnostic marker for distinguishing between AML, B-ALL and T-ALL comprises a pair of primers specific to (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L.

A DNA chip kit for detecting a diagnostic marker for distinguishing between AML, B-ALL and T-ALL comprises a base plate onto which cDNA corresponding to (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L, or fragments thereof.

An ELISA kit for detecting a diagnostic marker for distinguishing between AML, B-ALL and T-ALL comprises an antibody specific to (a) (i) the CITED2 protein, or (ii) the CITED2 protein and one or more proteins selected from among MGST1, BIN2, RAB32, ICAM-3, PXN, PPGB and TAF15; (b) (i) the TCL1A protein, or (ii) the TCL1A protein and one or more proteins selected from among CD19, INSR, OFD1, AKR1B1, CD79B and UHRF1; and (c) (i) the TCF7 protein, or (ii) the TCF7 protein and one or more proteins selected from among TRB, TRGC2, NK4 and CHC1L.

In still another aspect, the present invention relates to a composition for detecting a diagnostic marker for AML, comprising a pair of primers specific to (i) the CITED2 gene, or (ii) the CITED2 gene and one or more genes selected from among MGST1, BIN2, RAB32, ICAM-3, PXN, PPGB and TAF15.

The “primer”, as used herein, refers to a short nucleic acid sequence having a free 3′ hydroxyl group, which is able to form base-pairing interaction with a complementary template and serves as a starting point for replicating the template strand. A primer is able to initiate DNA synthesis in the presence of a reagent for polymerization (i.e., DNA polymerase or reverse transcriptase) and four different nucleoside triphosphates at suitable buffers and temperature. The primers of the present invention, specific to each of the marker genes, are sense and antisense nucleic acids having a sequence of 7 to 50 nucleotides. The primer may have additional properties that do not change the ability of the primer to serve as an origin for DNA synthesis.

The primers of the present invention may be chemically synthesized using a phosphoramidite solid support method or other widely known methods. These nucleic acid sequences may also be modified using any means known in the art. Non-limiting examples of such modifications include methylation, capsulation, replacement of one or more native nucleotides with analogues thereof, and inter-nucleotide modifications, for example, modifications to uncharged conjugates (e.g., methyl phosphonate, phosphotriester, phosphoroamidate, carbamate, etc.) or charged conjugates (e.g., phosphorothioate, phosphorodithioate, etc.). Nucleic acids may contain one or more additionally covalent-bonded residues, which are exemplified by proteins (e.g., nucleases, toxins, antibodies, signal peptides, poly-L-lysine, etc.), intercalating agents (e.g., acridine, psoralene, etc.), chelating agents (e.g., metals, radioactive metals, iron, oxidative metals, etc.), and alkylating agents. The nucleic acid sequences of the present invention may also be altered using a label capable of directly or indirectly supplying a detectable signal. Examples of the label include radioisotopes, fluorescent molecules and biotin.

Preferably, the composition for detecting an AML diagnostic marker is a composition for detecting CITED2 and MGST1 diagnostic markers, and includes two pairs of primers, one primer pair corresponding to SEQ ID Nos. 1 and for amplifying CITED2 and the other primer pair corresponding to SEQ ID Nos. 3 and 4 for amplifying MGST1. The composition may further include SEQ ID Nos. 13 and 14 for amplifying a control gene, RRAGB, and SEQ ID Nos. 15 and 16 for amplifying another control gene, DCK.

In still another aspect, the present invention relates to a composition for detecting a diagnostic marker for AML, comprising an antibody specific to (i) the CITED2 protein, or (ii) the CITED2 protein and one or more proteins selected from among MGST1, BIN2, RAB32, ICAM-3, PXN, PPGB and TAF15.

The term “antibody”, as used herein, refers to a specific protein molecule that indicates an antigenic region. With respect to the objects of the present invention, an “antibody” binds specifically to a marker protein, and includes polyclonal antibodies, monoclonal antibodies and recombinant antibodies.

Antibody production using the AML marker proteins identified as described above may be easily carried out using techniques widely known in the art.

Polyclonal antibodies may be produced using a method widely known in the art, which includes injecting the AML marker protein antigen into an animal and collecting blood samples from the animal to obtain sera containing antibodies. Such polyclonal antibodies may be prepared from a certain animal host, such as goats, rabbits, sheep, monkeys, horses, pigs, cows and dogs.

Monoclonal antibodies may be prepared by a method widely known in the art, such as a hybridoma method (see, Kohler and Milstein (1976) European Journal of Immunology 6:511-519), or a phage antibody library technique (Clackson et al., Nature, 352:624-628, 1991; Marks et al., J. Mol. Biol., 222:58, 1-597, 1991).

The hybridoma method employs cells from an immunologically suitable host animal injected with an AML diagnostic marker protein as an antigen, such as mice, and a cancer or myeloma cell line as another group. Cells of the two groups are fused with each other by a method widely known in the art, for example, using polyethylene, and antibody-producing cells are propagated using a standard tissue culture method. After uniform cell colonies are obtained by subcloning using a limited dilution technique, hybridomas capable of producing an antibody specific for the AML diagnostic marker protein are cultivated in large scale in vitro or in vivo according to a standard technique. Monoclonal antibodies produced by the hybridomas may be used in an unpurified form, but are preferably used after being purified through a method widely known in the art.

The phage antibody library method includes constructing a single-chain variable fragment (scFv) phage antibody library in vitro by obtaining genes for antibodies to a variety of intracellular AML protein markers and expressing them in a fusion protein form on the surface of phages, and isolating monoclonal antibodies from the library.

In addition, the antibodies of the present invention include complete forms, each of which consist of two full-length light chains and two full-length heavy chains, as well as functional fragments of antibody molecules. The functional fragments of antibody molecules refer to fragments retaining at least an antigen-binding function, and include Fab, F(ab′), F(ab′)₂ and Fv.

Preferably, the composition for detecting an AML diagnostic marker is a composition for detecting CITED2 and MGST1 diagnostic markers, and includes a CITED2-specific antibody and an MGST1-specific antibody. The composition may further include an RRAGB-specific antibody and a DCK-specific antibody.

In still another aspect, the present invention relates to a composition for detecting a diagnostic marker for B-ALL, comprising a pair of primers specific to (i) the TCL1A gene, or (ii) the TCL1A gene and one or more genes selected from among CD19, INSR, OFD1, AKR1B1, CD79B and UHRF1.

Preferably, the composition for detecting a B-ALL diagnostic marker is a composition for detecting TCL1A and CD19 diagnostic markers, and includes two pairs of primers, one primer pair corresponding to SEQ ID Nos. 5 and 6 for amplifying TCL1A and the other primer pair corresponding to SEQ ID Nos. 7 and 8 for amplifying CD19. The composition may further include SEQ ID Nos. 13 and 14 for amplifying a control gene, RRAGB, and SEQ ID Nos. 15 and 16 for amplifying another control gene, DCK.

In still another aspect, the present invention relates to a composition for detecting a diagnostic marker for B-ALL, comprising an antibody specific to (i) the TCL1A protein, or (ii) the TCL1A protein and one or more proteins selected from among CD19, INSR, OFD1, AKR1B1, CD79B and UHRF1.

Preferably, the composition for detecting a B-ALL diagnostic marker is a composition for detecting TCL1A and CD19 diagnostic markers, and includes a TCL1A-specific antibody and a CD19-specific antibody. The composition may further include an RRAGB-specific antibody and a DCK-specific antibody.

In still another aspect, the present invention relates to a composition for detecting a diagnostic marker for T-ALL, comprising a pair of primers specific to (i) the TCF7 gene, or (ii) the TCF7 gene and one or more genes selected from among TRB, TRGC2, NK4 and CHC1L.

Preferably, the composition for detecting a T-ALL diagnostic marker is a composition for detecting TCF7 and TRB diagnostic markers, and includes two pairs of primers, one primer pair corresponding to SEQ ID Nos. 9 and 10 for amplifying TCF7, and the other primer pair corresponding to SEQ ID Nos. 11 and 12 for amplifying TRB. The composition may further include SEQ ID Nos. 13 and 14 for amplifying a control gene, RRAGB, and SEQ ID Nos. 15 and 16 for amplifying another control gene, DCK.

In still another aspect, the present invention relates to a composition for detecting a diagnostic marker for T-ALL, comprising an antibody specific to (i) the TCF7 gene, or (ii) the TCF7 gene and one or more genes selected from among TRB, TRGC2, NK4 and CHC1L.

Preferably, the composition for detecting a T-ALL diagnostic marker is a composition for detecting TCF7 and TRB diagnostic markers, and includes a TCF7-specific antibody and a TRB-specific antibody. The composition may further include an RRAGB-specific antibody and a DCK-specific antibody.

In still another aspect, the present invention relates to a composition for detecting a diagnostic marker for distinguishing between AML, B-ALL and T-ALL, comprising a pair of primers specific to (a) (i) the CITED2 gene, or (ii) the CITED2 gene and one or more genes selected from among MGST1, BIN2, RAB32, ICAM-3, PXN, PPGB and TAF15; (b) (i) the TCL1A gene, or (ii) the TCL1A gene and one or more genes selected from among CD19, INSR, OFD1, AKR1B1, CD79B and UHRF1; and (c) (i) the TCF7 gene, or (ii) the TCF7 gene and one or more genes selected from among TRB, TRGC2, NK4 and CHC1L.

Preferably, the above composition includes pairs of primers specific to the CITED2 and MGST1 genes, the TCL1A and CD19 genes, and the TCF7 and TRB genes. A pair of primers for amplifying CITED2 is represented by SEQ ID Nos. 1 and 2. A pair of primers for amplifying MGST1 is represented by SEQ ID Nos. 3 and 4. A pair of primers for amplifying TCL1A is represented by SEQ ID Nos. 5 and 6. A pair of primers for amplifying CD19 is represented by SEQ ID Nos. 7 and 8. A pair of primers for amplifying TCF7 is represented by SEQ ID Nos. 9 and 10. A pair of primers for amplifying TRB is represented by SEQ ID Nos. 11 and 12. The composition may further include SEQ ID Nos. 13 and 14 for amplifying a control gene, RRAGB, and SEQ ID Nos. 15 and 16 for amplifying another control gene, DCK.

In still another aspect, the present invention relates to a composition for detecting a diagnostic marker for distinguishing between AML, B-ALL and T-ALL, comprising an antibody specific to (a) (i) the CITED2 protein, or (ii) the CITED2 protein and one or more proteins selected from among MGST1, BIN2, RAB32, ICAM-3, PXN, PPGB and TAF15; (b) (i) the TCL1A protein, or (ii) the TCL1A protein and one or more proteins selected from among CD19, INSR, OFD1, AKR1B1, CD79B and UHRF1; and (c) (i) the TCF7 protein, or (ii) the TCF7 protein and one or more proteins selected from among TRB, TRGC2, NK4 and CHC1L.

Preferably, the above composition includes antibodies specific to the CITED2 and MGST1 proteins, the TCL1A and CD19 proteins, and the TCF7 and TRB proteins.

A better understanding of the present invention may be obtained through the following examples which are set forth to illustrate, but are not to be construed as the limit of the present invention.

### Example 1

### Evaluation of Gene Expression in Bone Marrow Cells of AML and ALL Patients Using DNA Chip Assay

<1-1> RNA Isolation from Bone Marrow Cells

RNA was isolated from bone marrow specimens from 82 AML patients, 23 B-ALL patients and two T-ALL patients, as follows. 1 ml of each bone marrow specimen was mixed with 5 ml of a TriZol reagent (InVitrogen, Cat. No. 15596-018), and cells were disrupted for 1 min using a tissue homogenizer. Then, total RNA was isolated according to the manufacturer's protocol for the TriZol reagent. The isolated RNA was further purified to increase purity using an RNeasy kit (Qiagen, Cat. No. 74106) according to the manufacturer's protocol.

### <1-2> Quantitative Analysis of the Isolated RNA

The concentration of the isolated RNA was determined by measuring absorbance at 260 nm using a spectrophotometer.

### <1-3> Preparation of Reference RNA

RNA was isolated from cell lines originated from blood cells, and used as reference RNA to be hybridized along with the RNA isolated from bone marrow cells to a DNA chip. RNA was isolated from seven cell lines purchased from the Korean Cell Line Bank (http;//cellbank.snu.ac.kr), HL-60, K-562, CCRF-CEM, CCRF-HSB-2, CEM-CM3, Molt-4 and THP-1, according to the same method as in <1-1>. The isolated RNA samples were quantified, mixed in equal amounts, and used as reference RNA.

### <1-4> Preparation of DNA Chip

A 16K human cDNA chip containing 15,972 cDNA probes (Vivian G. Cheung et al., Nature Genetics, Making and reading microarrays, 1999 Jan. 21: 15-19; Microarray Biochip Technology, Mark Schena, 2000, Eaton Publishing) was used. The cDNA chip was prepared as follows. In brief, plasmid DNA was isolated from a bacterial stock containing plasmids into which cDNA had been cloned, and PCR was carried out using the isolated plasmid DNA as a template. To use the amplified cDNA as a probe, impurities were removed using a PCR Clean-Up Kit. The purified cDNA was dissolved in a spotting solution containing 50% DMSO to yield a final concentration of 100 to 200 ng/μl, spotted onto GAPS II slides (Corning, Cat. No. 40006), and irradiated with a suitable amount of UV light to immobilize it, thereby yielding the 16K human cDNA chip.

### <1-5> DNA Chip Assay and Quantification of Gene Expression

10 μg of the RNA isolated from bone marrow specimens and the reference RNA were reverse transcribed in the presence of aminoallyl-dUTP, and the synthesized cDNA was coupled to Cy5 and Cy3 monoester dyes, respectively. The labeled RNA was purified using a PCR Clean-Up Kit, and hybridized with the DNA chip for more than 16 hrs. After hybridization, the DNA chip was washed with a washing solution containing SSC to eliminate non-specific hybridizations. The washed DNA chip was scanned using a confocal laser scanner (Perkin Elmer, Scanarray Lite), and the obtained fluorescent data present at each spot were saved as TIFF images. The TIFF images were quantified with GenePix 3.0 (Axon Instruments) to quantify the fluorescence intensity at each spot. Quantitative results obtained from GenePix 3.0 were normalized using the ‘lowess’ function supplied by the S-plus statistical package (InSightful) according to a method suggested by Yang et al. (Nucleic Acids Res 2002, 30:e15).

### Example 2

### Selection of Genes Expressed at Different Levels in AML, B-ALL, T-ALL and Control Genes from DNA Chip Results

For 15,972 probes present in the DNA chip used, a t-test was conducted for a significance level of p<10⁻⁶ so as to select genes which are expressed in different levels between AML, B-ALL and T-ALL specimens. Since the t-test was repeated 15,972 times, 0.02 false positives were expected to be expressed, given a significance level of p<10⁻⁶. Thus, all genes selected were genes that actually exhibited different expression levels. The t-test resulted in the selection of 268 genes differently expressed in AML, B-ALL and T-ALL specimens.

To select genes for diagnosing AML, B-ALL and T-ALL using RT-PCR, ten or fewer genes for each of the leukemia types were selected from the 268 primarily selected genes in consideration of p values obtained in the t-test and the difference in gene expression levels between AML, B-ALL and T-ALL specimens (Tables 1, 2 and 3).

TABLE 1
Genes highly expressed specifically in AML

| | | Fold | | GenBank | | |
|---|---|---|---|---|---|---|
| | | difference | | Accession | Unigen | Gene |
| t value | p value | (AML/ALL) | Description | number | Cluster ID | symbol |
| −8.39 | p < | 7.56 | “RAB32, member RAS oncogene | AA057378 | Hs.32217 | RAB32 |
| | 0.000001 | | family” | | | |
| −8.25 | p < | 4.11 | bridging integrator 2 | AI189483 | Hs.14770 | BIN2 |
| | 0.000001 | | | | | |
| −7.3 | p < | 3.30 | “Homo sapiens, Similar to intercellular | AA479188 | Hs.353214 | ICAM3 |
| | 0.000001 | | adhesion molecule 3, clone | | | |
| | | | IMAGE: 5205468, mRNA” | | | |
| −7.14 | p < | 2.69 | paxillin | AA430574 | Hs.102497 | PXN |
| | 0.000001 | | | | | |
| −7.06 | p < | 2.85 | protective protein for | AA916327 | Hs.118126 | PPGB |
| | 0.000001 | | beta-galactosidase (galactosialidosis) | | | |
| −6.99 | p < | 2.49 | “ceroid-lipofuscinosis, neuronal 2, late | AA664004 | Hs.20478 | CLN2 |
| | 0.000001 | | infantile (Jansky-Bielschowsky | | | |
| | | | disease)” | | | |
| −6.86 | p < | 4.45 | “TAF15 RNA polymerase II, TATA | AA857343 | Hs.381044 | TAF15 |
| | 0.000001 | | box binding protein (TBP)-associated | | | |
| | | | factor, 68 kDa” | | | |
| −6.83 | p < | 3.32 | “Cbp/p300-interacting transactivator, | AA115076 | Hs.82071 | CITED2 |
| | 0.000001 | | with Glu/Asp-rich carboxy-terminal | | | |
| | | | domain, 2” | | | |
| −6.56 | p < | 4.20 | microsomal glutathione S-transferase 1 | AA495936 | Hs.389700 | MGST1 |
| | 0.000001 | | | | | |

TABLE 2
Genes highly expressed specifically in B-ALL

| | | Fold | | GenBank | | |
|---|---|---|---|---|---|---|
| | | difference | | Accession | Unigen | Gene |
| t-value | p value | (B-ALL/AML) | Description | number | Cluster ID | symbol |
| 6.36 | p < | 5.82 | CD79A antigen | M80462 | Hs.79630 | CD79A |
| | 0.000001 | | (immunoglobulin-associated alpha) | | | |
| 6.79 | p < | 3.05 | Insulin receptor | AA001614 | Hs.438669 | INSR |
| | 0.000001 | | | | | |
| 6.85 | p < | 4.74 | CD19 antigen | AI356451 | Hs.96023 | CD19 |
| | 0.000001 | | | | | |
| 6.89 | p < | 4.64 | Oral-facial-digital syndrom 1 | AA173595 | Hs.6483 | OFD1 |
| | 0.000001 | | | | | |
| 7.01 | p < | 2.83 | “aldo-keto reductase family 1, | AA701963 | Hs.75313 | AKR1B1 |
| | 0.000001 | | member B1 (aldose reductase)” | | | |
| 7.32 | p < | 6.77 | “membrane metallo-endopeptidase | R98936 | Hs.1298 | MME |
| | 0.000001 | | (neutral endopeptidase, | | | |
| | | | enkephalinase, CALLA, CD10)” | | | |
| 7.59 | p < | 5.09 | CD79B antigen | R72079 | Hs.89575 | CD79B |
| | 0.000001 | | (immunoglobulin-associated beta) | | | |
| 7.76 | p < | 5.70 | “ubiquitin-like, containing PHD | N59762 | Hs.108106 | UHRF1 |
| | 0.000001 | | and RING finger domains, 1” | | | |
| 10.14 | p < | 4.83 | T-cell leukemia/lymphoma 1A | NM_021966 | Hs.2484 | TCL1A |
| | 0.000001 | | | | | |

TABLE 3
Genes highly expressed specifically in T-ALL

| | | | | GenBank | | |
|---|---|---|---|---|---|---|
| | | Fold difference | | Accession | UniGene | Gene |
| t-value | p value | (B-ALL/T-ALL) | Description | number | Cluster ID | symbol |
| −24.38 | p < 0.000001 | 19.64 | CD3D antigen, delta | AA919102 | Hs.95327 | CD3D |
| | | | polypeptide (TiT3 complex) | | | |
| −15.81 | p < 0.000001 | 17.83 | T cell receptor beta locus | X00437 | Hs.419777 | TRB |
| −9.61 | p < 0.000001 | 16.97 | mal, T-cell differentiation | X76220 | Hs.80395 | MAL |
| | | | protein | | | |
| −16.31 | p < 0.000001 | 10.22 | transcription factor 7 (T-cell | X59871 | Hs.169294 | TCF7 |
| | | | specific, HMG-box) | | | |
| −19.63 | p < 0.000001 | 7.86 | lymphocyte-specific protein | U23852 | Hs.1765 | LCK |
| | | | tyrosine kinase | | | |
| −7.95 | p < 0.000001 | 7.84 | SH2 domain protein 1A, | AL023657 | Hs.151544 | SH2D1A |
| | | | Duncan's disease | | | |
| | | | (lymphoproliferative syndrome) | | | |
| −11.99 | p < 0.000001 | 9.46 | T cell receptor delta locus | X73617 | Hs.2014 | TRD@ |
| −6.85 | p < 0.000001 | 6.67 | T cell receptor gamma constant 2 | M30894 | Hs.385086 | TRGC2 |
| −7.91 | p < 0.000001 | 6.30 | natural killer cell transcript 4 | AA631972 | Hs.943 | NK4 |
| −7.89 | p < 0.000001 | 6.10 | linker for activation of T cells | AJ223280 | Hs.437775 | LAT |
| −6.35 | p < 0.000001 | 4.98 | chromosome condensation 1- | AF060219 | Hs.27007 | CHC1L |
| | | | like | | | |

In addition, to select genes that were expressed at constant levels in all specimens, the gene expression variance for each gene was estimated in all specimens, and the seven genes having the lowest variance in gene expression were selected (Table 4).

TABLE 4
Genes expressed at constant levels in all specimens

| | | GenBank | | |
|---|---|---|---|---|
| | | Accession | UniGene | Gene |
| Variance | Description | number | Cluster ID | symbol |
| 0.002131 | nebulette | AI700281 | Hs.5025 | NEBL |
| 0.002987 | deoxycytidine kinase | AI760771 | Hs.709 | DCK |
| 0.003067 | GTP-binding protein ragB | AA234339 | Hs.50282 | RRAGB |
| 0.003248 | ORF | AA814214 | Hs.351296 | LOC51035 |
| 0.003296 | Homo sapiens transcribed sequence | AI051950 | Hs.445321 | |
| | with weak similarity to protein | | | |
| | ref: NP_073606.1 (H. sapiens) | | | |
| | hypothetical protein FLJ21868 | | | |
| | [Homo sapiens] | | | |
| 0.003766 | Sapiens mRNA; cDNA DKFZp566B213 | AI244975 | Hs.194051 | |
| | (from clone DKFZp566B213) | | | |
| 0.003878 | platelet-derived growth factor alpha | AI625002 | Hs.37040 | PDGFA |
| | polypeptide | | | |

### Example 3

### Selection of AML, B-ALL and T-ALL-Specific Diagnostic Markers and Control Genes Using RT-PCR

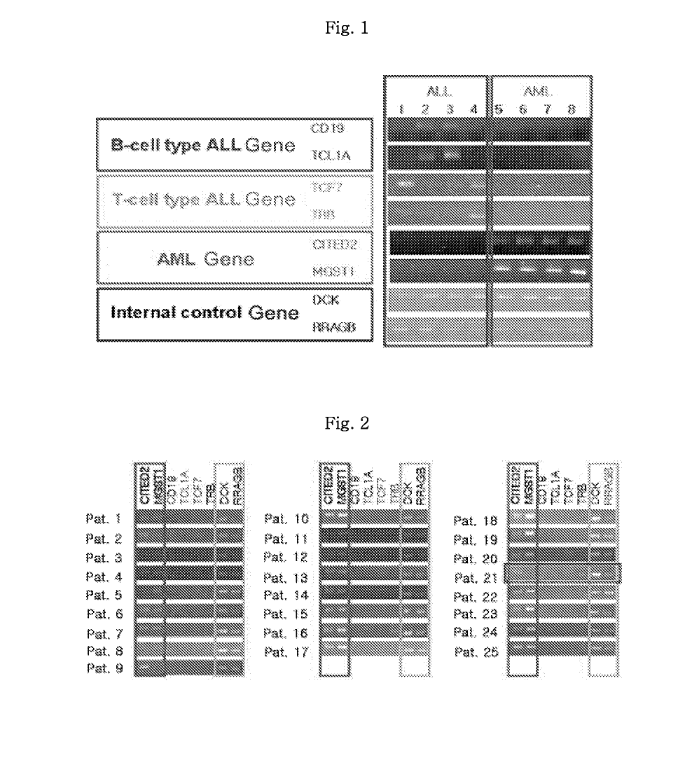

In order to determine whether AML and ALL could be distinguished by the differential expression of the candidate diagnostic marker genes selected in Example 2, expression levels of the selected genes were examined in four AML specimens, two T-ALL specimens and two B-ALL specimens. RT-PCR was carried out as follows. 5 μg of RNA was reverse transcribed in a 20 μl reaction volume, and was diluted with distilled water to 100 μg. Using 2 μl of the diluted RT-PCR product as a template, 25 cycles of PCR were carried out with pairs of primers specific to eight marker genes in a 25-μl reaction volume. 8 μl of each PCR product was electrophoresed on a 2% agarose gel containing 0.5 μg/ml of EtBr, and DNA bands were observed under UV light. Among the primarily selected genes, genes most specific to AML, B-ALL and T-ALL and two control genes expressed at constant levels in all specimens were selected. FIG. 1 shows the results of RT-PCR of the eight selected genes.

### Example 4

### Diagnosis of AML and ALL in 57 Acute Leukemia Bone Marrow Cells Using the Eight Diagnostic Marker Genes

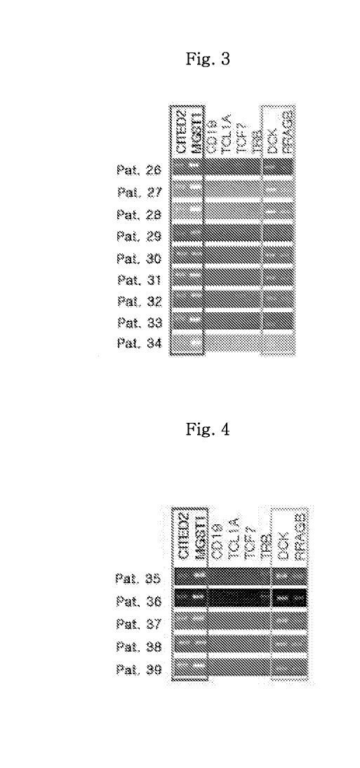

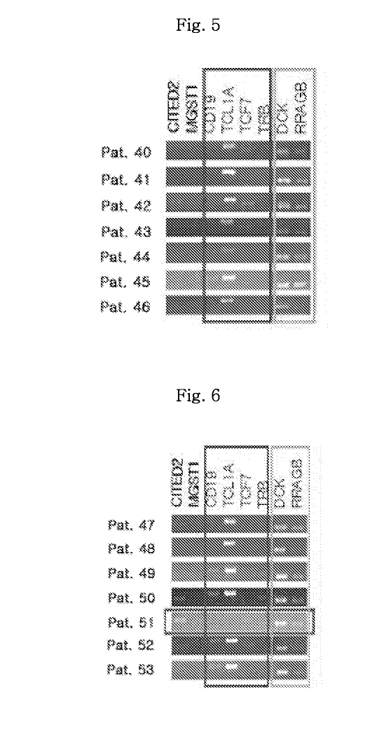

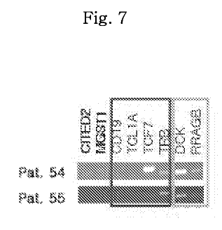

The expression of the eight diagnostic marker genes selected in Example 3 was examined in additional 41 AML specimens and 16 ALL specimens. Acute leukemia is known to have characteristic chromosomal abnormalities. For example, in AML, the most frequent chromosomal abnormalities include t(8;21), t(15;17), and inv(16). When found in ALL, the t(9;22) chromosomal abnormality indicates a very poor patient prognosis. To determine whether the diagnostic marker genes selected in Example 3 could be applied to acute leukemia having several chromosomal abnormalities, the expression of the eight diagnostic marker genes was examined in specimens having several chromosomal abnormalities. The results are given in FIGS. 2 to 7, wherein the expression of the eight diagnostic marker genes was examined in AML specimens having normal chromosomes (FIG. 2), AML specimens having a t(15;17) chromosomal abnormality (FIG. 3), AML specimens having a t(8;21) chromosomal abnormality (FIG. 4), B-ALL specimens having normal chromosomes (FIG. 5), B-ALL specimens having a t(9;22) chromosomal abnormality (FIG. 6), and T-ALL specimens (FIG. 7).

As a result, 38 of 39 AML specimens expressed one or more AML-specific diagnostic marker genes, but did not express ALL-specific marker genes. The one remaining AML specimen expressed both AML and ALL marker genes. Also, 15 of 16 ALL specimens expressed one or more ALL-specific diagnostic marker genes, but did not express AML-specific marker genes. The one remaining ALL specimen expressed both ALL and AML marker genes. The 55 acute leukemia specimens all expressed one or more control genes. These results indicate that distinction between AML, B-ALL and T-ALL is possible by detecting the expression of the eight marker genes selected in Example 3.

Since 38 of the 39 AML specimens and 15 of the 16 ALL specimens were correctly diagnosed, the sensitivity of the present diagnosis was 97.4% for AML diagnosis and 93.8% for ALL diagnosis, thus giving an overall average sensitivity of 96.4%. Also, since 15 of the 16 specimens not having AML were not diagnosed with AML, and 38 of the 39 specimens not having ALL were not diagnosed with ALL, the specificity of the present diagnosis was 93.8% for AML diagnosis and 97.4% for ALL diagnosis, thus giving an overall average specificity of 96.4% (Table 5).

TABLE 5
Sensitivity and specificity of diagnosis

| | | | | Specimens | | |
|---|---|---|---|---|---|---|
| | | | | whose | | |
| | | Specimens | Specimens | diagnosis | | |
| Leukemia | Total No. of | diagnosed | diagnosed | was | | |
| type | specimens | with AML | with ALL | impossible | Sensitivity | Specificity |
| AML | 39 | 38 | 0 | 1 | 38/39 | 15/16 |
| | | | | | (97.4%) | (93.8%) |
| ALL | 16 | 0 | 15 | 1 | 15/16 | 38/39 |
| | | | | | (93.8%) | (97.4%) |
| Total | 55 | 38 | 15 | 2 | 53/55 | 53/55 |
| | | | | | (96.4) | (96.4%) |

### INDUSTRIAL APPLICABILITY

As described hereinbefore, the different types of acute leukemia can be simply and accurately diagnosed using the present method of distinguishing between AML, B-ALL and T-ALL, which is based on detecting mRNA and protein levels of the leukemia diagnostic markers.

1-19. (canceled)

20. A method for diagnosis of acute myeloid leukemia, comprising the steps of;
(a) measuring mRNA or protein levels of (i) CITED2 (SEQ ID:17) gene or (ii) CITED2 (SEQ ID:17) gene and one or more genes selected from among MGST1 (SEQ ID:18), BIN2 (SEQ ID:19), RAP32 (SEQ ID:20), ICAM-3 (SEQ ID:21), PXN (SEQ ID:22), a fragment of PPGB (SEQ ID:23), and TAF15 (SEQ ID:24) in a biological sample from a patient suspected of having leukemia; and
(b) comparing mRNA or protein levels of the sample from a patient with those of a normal control sample to determine the increase in mRNA or protein levels.

21. The method as set forth in claim 20, measuring mRNA or protein levels of the CITED2 (SEQ ID:17) and MGST1 (SEQ ID:18) genes in a biological sample from a patient suspected of having leukemia.

22. The method as set forth in claim 20, wherein the measuring mRNA level is performed by the method selected from the group consisting of RT-PCR, competitive RT-PCR, real-time RT-PCR, RNase protection assay (RPA), Northern blotting and DNA chip assay.

23. The method as set forth in claim 20, wherein the measuring protein level is performed by the method selected from the group consisting of Western blotting, enzyme linked immunosorbent assay (ELISA), radioimmunoassay (RIA), radioimmunodiffusion, Ouchterlony immunodiffusion, rocket immunoelectrophoresis, immunohistostaining, immunoprecipitation assay, complement fixation assay, FACS and protein chip assay.
